(19) United States
(12) Patent Application Publication
(10) Pub. No.: US 2015/0223987 A1
(43) Pub. Date: Aug. 13, 2015

(54) PROTECTIVE EAR APPLIANCE AND A COMPOSITION FOR STICKING RHINESTONES ON THE APPLIANCE

(71) Applicant: Tami Cimperman, Port St. Lucie, FL (US)

(72) Inventor: Tami Cimperman, Port St. Lucie, FL (US)

(21) Appl. No.: 14/697,293

(22) Filed: Apr. 27, 2015

(51) Int. Cl. A61F 11/14; C09J 127/04; C09J 5/00

(52) U.S. Cl. CPC A61F 11/14; C09J 5/00; C09J 127/04; C09J 2427/00; C09J 2203/10; C09J 2489/006

(57) ABSTRACT

The present invention discloses a method for fabricating an earband/earmuff, according to an embodiment herein. The first step for fabricating an earband/earmuff is by fixing or adhering the “O” shaped rings. The next step is coating and sewing pu leather around the plastic band. Further obtaining a flat and smooth surface on plastic band after coating with pu leather. The next step is adhering a plurality of rhinestones/charms/custom emblem or logos on earband/earmuff with a glue. The next step is testing the earband/earmuff for strength by subjecting to a temperature below 0° C. temperature and above 90° C. temperature. The rhinetsones do not separate from the earband because of the adhesive in different temperature ranges. A glue “E 6000®” is applied for adhering the rhinestones on the smooth surface of the earband or earmuff. The glue “E 6000®” comprises of tetrachloroethylene. The quantity of tetracholoroethylene is present in 60-100 weight %.

### BACKGROUND

1. Technical Field

The embodiments herein generally relate to a protective device. The embodiments herein generally relate to a protective ear appliance. The embodiments herein more particularly relates to an earmuff or ear band.

2. Description of the Related Art

Earbands or earmuffs are objects designed to cover a person's ears for protection. Generally the ear bands consist of a thermoplastic or metal head band that fits over the top or back of the head and a pad at each end. These pads cover the external ears.

There are two basic kinds of ear bands/earmuffs. They are 1) thermal ear bands and 2) acoustic ear bands. The thermal ear bands are worn in cold environments to keep a person's ears warm with pads. The pads are made of cloth or fur. The acoustic earmuffs are also known as ear defenders where the pads covering the ears are lined with sound defending material. The acoustic ear bands are like thermal ear bands and headphones in appearance. The acoustic ear bands are worn as hearing protection. The acoustic earmuffs are carried on a head band or clipped onto the sides of a hard hat for use on construction sites.

The ear bands or earmuffs protect the wearer from extreme noises, pollution and cold.

The ear bands or earmuffs are decorated in many ways. One such way is to fix rhinestones. Rhinestones make the ear bands attractive and lucrative. Rhinestones are diamond stimulant made from rock crystal, glass or acrylic.

The earlier attempts were made where larger stones are glued individually to a very narrow plastic band. The uneven or rough plastic band caused uneven application of the rhinestones and glue that was used to hold the rhinestones in a place permanently. Also, the application of large rhinestones placed sporadically along a narrow band does not allow for multiple rhinestones or intricate rhinestone designs.

Hence there is a need for making a wider ear band with a smooth or even surface. Also there is a need for a glue composition for sticking or adding the rhinestones to secure the rhinestones on the ear band. Further there is a need for an ear band having flexibility and characteristics for fabrication. Further there is need to fabricate a fashionable product with intricate and uniform looks and design with a quality to withstand all temperature conditions.

The above mentioned shortcomings, disadvantages and problems are addressed herein and which will be understood by reading and studying the following specification.

### OBJECTIVES OF THE EMBODIMENTS

The primary objective of the embodiment herein is to fabricating an ear band with a smooth and even surface with externally adhered rhinestones.

Another object of the embodiment herein is to adhere and secure the rhinestones on ear band with a glue composition.

Yet another object of the embodiment herein is to provide an ear band with “one size fits all” principle.

Yet another object of the embodiment herein is to provide an ear band with externally adhered rhinestones which do not fall or become loose in various temperature ranges.

These objects and the other advantages of the embodiments herein will become readily apparent from the following detailed description taken in conjunction with the accompanying drawings.

### SUMMARY

The various embodiments herein provide an ear band with a smooth and even surface with externally adhered rhinestones. The rhinestones are adhered and secured on ear band with a glue composition. Also the externally adhered rhinestones do not fall or become loose in various temperature ranges.

According to one embodiment herein, the method for fabricating an ear band/earmuff comprises the following steps. The first step is fixing or adhering the “O” shaped rings on two ends of a plastic band. Further pu leather is coated or sewed around the plastic band. A flat and a smooth surface on the plastic band is obtained after coating with pu leather. The next step is adhering a plurality of articles on the ear band/earmuff with a glue. The ear band/earmuff is cured after adhering the plurality of articles with the glue for a preset curation time. The ear band or earmuff is subjected to a plurality of temperature levels for testing a adhesive strength.

According to one embodiment herein, the glue is a craft adhesive or an article adhesive, and the adhesive or glue is E6000®. The plurality of articles for adhering on the surface of ear band or earmuff are selected from a group comprising of a rhinestone, a charm, a customized emblem and a logo. The ear band or earmuff is subjected to a predetermined temperature range, and wherein the temperature range is 0° C.-90° C. The preset curation time after the glue or adhesive is applied to an article is 20 minutes. The plurality of articles are adhered on the ear band/earmuff with a thin adhesive film or a thick adhesive film. The full curation time of a thin adhesive film is 24 hours. A full curation time of a thick adhesive film is 48-72 hours.

According to one embodiment herein, the glue or adhesive composition for adhering plurality of articles to an ear band/earmuff comprises tetrachloroethylene. The tetrachloroethylene is a cholorocarbon. The tetrachloroethylene has a formula Cl2C═CCl2.

According to one embodiment herein, the tetrachloroethylene is present in a predetermined quantity and wherein the predetermined quantity is 60-100 weight %. The glue composition has a sweet odour. The glue composition is non flammable. The glue composition has a density of 11.0±0.1 lbs/gal. The glue composition has a viscosity of 100,000 cps.

According to one embodiment herein, the curation time after the glue or adhesive is applied to an article is 20 minutes. The glue is applied as a thin adhesive film or a thick adhesive film. A full curation time of a thin adhesive film is 24 hours, and the full curation time of a thick adhesive film is 48-72 hours. The glue composition is resistant to a plurality of dilute acids and bases.

According to one embodiment herein, the dilute acids and bases are selected from a group comprising of acetic acid (5% and 10% by volume), ammonium hydroxide (3.4% by volume), ammonium nitrate (50% by volume), beer (3.2% by volume), boric acid (3.1% by volume), distilled water, hydraulic oil, lactic acid (3.8% by volume), milk, motor oil, nitric acid (10% and 20% by volume), oxalic acid (3.1% by volume), phosphoric acid (30% and 60% by volume), potassium hydroxide (3.4% by volume), sodium carbonate (2.7% by volume), sodium chloride (10%), sulfuric acid (3% and 10% by volume) and wine (20% alcohol by volume).

According to one embodiment herein, the ear band or earmuff is fabricated with a plastic band and pu leather. The pu leather is rigid and flexible. The plastic band has “0” shaped rings at both the ends. The plastic band is coated with pu leather. The pu leather is sewn completely over the plastic band. This provides a flat and smooth surface to adhere five rows of rhinestones.

According to one embodiment herein, the earband or earmuff is held to the ears of the wearer by the “O” shaped rings present on both ends of the plastic band. The “O” shaped rings are glued to both ends of the earband/earmuff. The “O” shaped rings make the earband “one size fits all”.

According to one embodiment herein, the pu leather coated plastic band provides a smooth and even surface for adhering plurality of rhinestones in various designs. A glue composition is used for adhering or fixing the rhinestones on the ear band. The glue adheres the rhinestones to the ear band strongly and rhinestones stay securely on the band.

After fixing the rhinestones on the surface of the ear bands, the rhinestones are tested in below 0° C. temperature and 90° C. humid temperature conditions. In these temperature conditions none of the rhinestones moved, became loose and fell off the ear band. The right combination of material thickness, glue and size of rhinestones enhances the ear band quality.

According to one embodiment herein, the materials used in fabrication of the earband makes the earband flexible. The flexibility of the earband allow to create plurality of intricate designs, application of logos, plurality of rhinestone designs, application of custom emblem/charms with logos. The size and shape of the rhinestones doesn't affect the curve of the earband. Also the size and shape of the rhinestones stick together without showing any excess glue or spacing in the earband. The size and shape of the rhinestones also allows maintenance of the earband curved shape without placing stress on particular stones that are placed in various susceptible stress points. Generally the stress causes removal of the rhinestones.

The charms are also fixed or adhered on the earband/earmuff. The earband with charm are customized based on the customer demand and to promote a specific team, logo or event. The metal charm is produced to specific size requirements. The metal charms like rhinestones do not create any stress points along the curvature of earband/earmuff. Hence in absence of stress rhinestones or charms do not separate from the earband or earmuff.

According to one embodiment herein, glue “E 6000®” is applied for adhering the rhinestones on the smooth surface of the earband or earmuff. The glue “E 6000®” comprises of tetrachloroethylene. The quantity of tetracholoroethylene is present in 60-100 weight %.

Tetracholoroethylene is also known under the systematic name tetracholoethene or percholoroethylene (“perc” or “PERC”). Tetrachloroethylene is cholorocarbon with a formula Cl₂C═CCl₂. Tetracholorethylene is a colorless liquid widely used for dry cleaning of fabrics, hence it is also called as “dry-cleaning fluid”. Tetrachloroethylene has a sweet odor detectable by most people at a concentration of 1 part per million (1 ppm).

The E6000 is a craft adhesive. The main component or ingredient is tetracholoroethylene. The tetrachloroethylene has no color or it is clear. Volatile organic compound (VOC) is present in a concentration of 2 grams/liter. The composition or ingredients of E 6000 is non flammable. The density of the E 6000 is 11.0±0.1 lbs/gal. The solids are present in the composition is 30% by weight and 40% by volume. The viscosity of E 6000 craft adhesive is 100,000 cps. After the adhesive is applied to an article the set time is 20 minutes. The full curation time of a thin film of adhesive is 24 hours. The full curation time of a thick film of adhesive is 48-72 hours. The storage temperature for adhesive is in a range of 50-80° F. (10 and 27° C.). The adhesive thickens when stored in a cold temperature. When the adhesive thickens then adhesive is thawed or kept in room temperature before use. The shelf life of un-opened adhesive is 2 years.

An adhesion test “180° peel strength test” is done to test the strength of E6000 to hold the adhered articles. The E6000 adhesive is applied on different materials aluminum, birch (wood), glass, polyvinyl chloride and steel. The adhesion test yield results of 35 pli, 40 pli, 34 pli, 38 pli and 45 pli for the different materials respectively. The pli is pounds per linear inch, a unit for defining the adhesion force.

The adhesive E6000®, exhibits high resistance to water, dilute acids and dilute bases. The films of E6000® are immersed in various chemicals for two weeks and the adhesive exhibited high weight gains of less than 2%. Also a loss of tensile strength less than 10% is observed. The dilute acids and dilute basis to which the adhesive E6000® is resistant are acetic acid (5% and 10% by volume), ammonium hydroxide (3.4% by volume), ammonium nitrate (50% by volume), beer (3.2% by volume), boric acid (3.1% by volume), distilled water, hydraulic oil, lactic acid (3.8% by volume), milk, motor oil, nitric acid (10% and 20% by volume), oxalic acid (3.1% by volume), phosphoric acid (30% and 60% by volume), potassium hydroxide (3.4% by volume), sodium carbonate (2.7% by volume), sodium chloride (10%), sulfuric acid (3% and 10% by volume) and wine (20% alcohol by volume).

The adhesive E6000®, gets dissolved in common solvents. Following are the solvents in which the adhesive E6000® gets dissolved: chevron solvent 1100, cholorothane, cyclohexane, gasoline, methylene chloride, percholoroethylene, propyl acetate, toluene, 1,1,1-tricholoethane.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flowchart indicating a method for fabricating an earband/earmuff, according to an embodiment herein.

Although the specific features of the embodiments herein are shown in some drawings and not in others. This is done for convenience only as each feature may be combined with any or all of the other features in accordance with the embodiments herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide an earband with a smooth and even surface with externally adhered rhinestones. The rhinestones are adhered and secured on earband with a glue composition. Also the externally adhered rhinestones do not fall or become loose in various temperature ranges.

According to one embodiment herein, the method for fabricating an earband/earmuff comprises the following steps. The first step is fixing or adhering the “O” shaped rings on two ends of a plastic band. Further pu leather is coated or sewed around the plastic band. A flat and a smooth surface on the plastic band is obtained after coating with pu leather. The next step is adhering a plurality of articles on the earband/earmuff with a glue. The earband/earmuff is cured after adhereing the plurality of articles with the glue for a preset curation time. The earband or earmuff is subjected to a plurality of temperature levels for testing a adhesive strength.

According to one embodiment herein, the glue is a craft adhesive or an article adhesive, and the adhesive or glue is E6000®. The plurality of articles for adhering on the surface of earband or earmuff are selected from a group comprising of a rhinestone, a charm, a customized emblem and a logo. The ear band or earmuff is subjected to a predetermined temperature range, and wherein the temperature range is 0° C.-90° C. The preset curation time after the glue or adhesive is applied to an article is 20 minutes. The plurality of articles are adhered on the earband/earmuff with a thin adhesive film or a thick adhesive film. The full curation time of a thin adhesive film is 24 hours. A full curation time of a thick adhesive film is 48-72 hours.

According to one embodiment herein, the glue or adhesive composition for adhering plurality of articles to an earband/earmuff comprises tetracholoroethylene. The tetrachloroethylene is a cholorocarbon. The tetracholoethylene has a formula Cl2C═CCl2.

According to one embodiment herein, the tetracholoroethylene is present in a predetermined quantity and wherein the predetermined quantity is 60-100 weight %. The glue composition has a sweet odor. The glue composition is non flammable. The glue composition has a density of 11.0±0.1 lbs/gal. The glue composition has a viscosity of 100,000 cps.

According to one embodiment herein, the curation time after the glue or adhesive is applied to an article is 20 minutes. The glue is applied as a thin adhesive film or a thick adhesive film. A full curation time of a thin adhesive film is 24 hours, and the full curation time of a thick adhesive film is 48-72 hours. The glue composition is resistant to a plurality of dilute acids and bases.

According to one embodiment herein, the dilute acids and bases are selected from a group comprising of acetic acid (5% and 10% by volume), ammonium hydroxide (3.4% by volume), ammonium nitrate (50% by volume), beer (3.2% by volume), boric acid (3.1% by volume), distilled water, hydraulic oil, lactic acid (3.8% by volume), milk, motor oil, nitric acid (10% and 20% by volume), oxalic acid (3.1% by volume), phosphoric acid (30% and 60% by volume), potassium hydroxide (3.4% by volume), sodium carbonate (2.7% by volume), sodium chloride (10%), sulfuric acid (3% and 10% by volume) and wine (20% alcohol by volume).

According to one embodiment herein, the earband or earmuff is fabricated with a plastic band and pu leather. The pu leather is rigid and flexible. The plastic band has “O” shaped rings at both the ends. The plastic band is coated with pu leather. The pu leather is sewn completely over the plastic band. This provides a flat and smooth surface to adhere five rows of rhinestones.

According to one embodiment herein, the earband or earmuff is held to the ears of the wearer by the “O” shaped rings present on both ends of the plastic band. The “O” shaped rings are glued to both ends of the earband/earmuff. The “O” shaped rings make the earband “one size fits all”.

According to one embodiment herein, the pu leather coated plastic band provides a smooth and even surface for adhering plurality of rhinestones in various designs. A glue composition is used for adhering or fixing the rhinestones on the earband. The glue adheres the rhinestones to the earband strongly and rhinestones stay securely on the band.

After fixing the rhinestones on the surface of the earbands, the rhinestones are tested in below 0° C. temperature and 90° C. humid temperature conditions. In these temperature conditions none of the rhinestones moved, became loose and fell off the earband. The right combination of material thickness, glue and size of rhinestones enhances the earband quality.

According to one embodiment herein, the materials used in fabrication of the earband makes the earband flexible. The flexibility of the earband allow to create plurality of intricate designs, application of logos, plurality of rhinestone designs, application of custom emblem/charms with logos. The size and shape of the rhinestones doesn't affect the curve of the earband. Also the size and shape of the rhinestones stick together without showing any excess glue or spacing in the earband. The size and shape of the rhinestones also allows maintenance of the earband curved shape without placing stress on particular stones that are placed in various susceptible stress points. Generally the stress causes removal of the rhinestones.

The charms are also fixed or adhered on the earband/earmuff. The earband with charm are customized based on the customer demand and to promote a specific team, logo or event. The metal charm is produced to specific size requirements. The metal charms like rhinestones do not create any stress points along the curvature of earband/earmuff. Hence in absence of stress rhinestones or charms do not separate from the earband or earmuff.

According to one embodiment herein, glue “E 6000®” is applied for adhering the rhinestones on the smooth surface of the earband or earmuff. The glue “E 6000®” comprises of tetrachloroethylene. The quantity of tetracholoroethylene is present in 60-100 weight %.

Tetracholoroethylen is also known under the systematic name tetracholoethene or percholoroethylene (“perc” or “PERC”). Tetrachloroethylene is cholorocarbon with a formula Cl₂C═CCl₂. Tetracholorethylene is a colorless liquid widely used for dry cleaning of fabrics, hence it is also called as “dry-cleaning fluid”. Tetrachloroethylene has a sweet odor detectable by most people at a concentration of 1 part per million (1 ppm).

The E6000 is a craft adhesive. The main component or ingredient is tetracholoroethylene. The tetrachloroethylene has no color or it is clear. Volatile organic compound (VOC) is present in a concentration of 2 grams/liter. The composition or ingredients of E 6000 is non flammable. The density of the E 6000 is 11.0±0.1 lbs/gal. The solids are present in the composition is 30% by weight and 40% by volume. The viscosity of E 6000 craft adhesive is 100,000 cps. After the adhesive is applied to an article the set time is 20 minutes. The full curation time of a thin film of adhesive is 24 hours.

The full curation time of a thick film of adhesive is 48-72 hours. The storage temperature for adhesive is in a range of 50-80° F. (10 and 27° C.). The adhesive thickens when stored in a cold temperature. When the adhesive thickens then adhesive is thawed or kept in room temperature before use. The shelf life of un-opened adhesive is 2 years.

A adhesion test “180° peel strength test” is done to test the strength of E6000 to hold the adhered articles. The E6000 adhesive is applied on different materials aluminum, birch (wood), glass, polyvinyl chloride and steel. The adhesion test yield results of 35 pli, 40 pli, 34 pli, 38 pli and 45 pli for the different materials respectively. The pli is pounds per linear inch, a unit for defining the adhesion force.

The adhesive E6000®, exhibits high resistance to water, dilute acids and dilute bases. The films of E6000® are immersed in various chemicals for two weeks and the adhesive exhibited high weight gains of less than 2%. Also a loss of tensile strength less than 10% is observed. The dilute acids and dilute basis to which the adhesive E6000® is resistant are acetic acid (5% and 10% by volume), ammonium hydroxide (3.4% by volume), ammonium nitrate (50% by volume), beer (3.2% by volume), boric acid (3.1% by volume), distilled water, hydraulic oil, lactic acid (3.8% by volume), milk, motor oil, nitric acid (10% and 20% by volume), oxalic acid (3.1% by volume), phosphoric acid (30% and 60% by volume), potassium hydroxide (3.4% by volume), sodium carbonate (2.7% by volume), sodium chloride (10%), sulfuric acid (3% and 10% by volume) and wine (20% alcohol by volume).

The adhesive E6000®, gets dissolved in common solvents. Following are the solvents in which the adhesive E6000® gets dissolved: chevron solvent 1100, cholorothane, cyclohexane, gasoline, methylene chloride, percholoroethylene, propyl acetate, toluene, 1,1,1-tricholoethane.

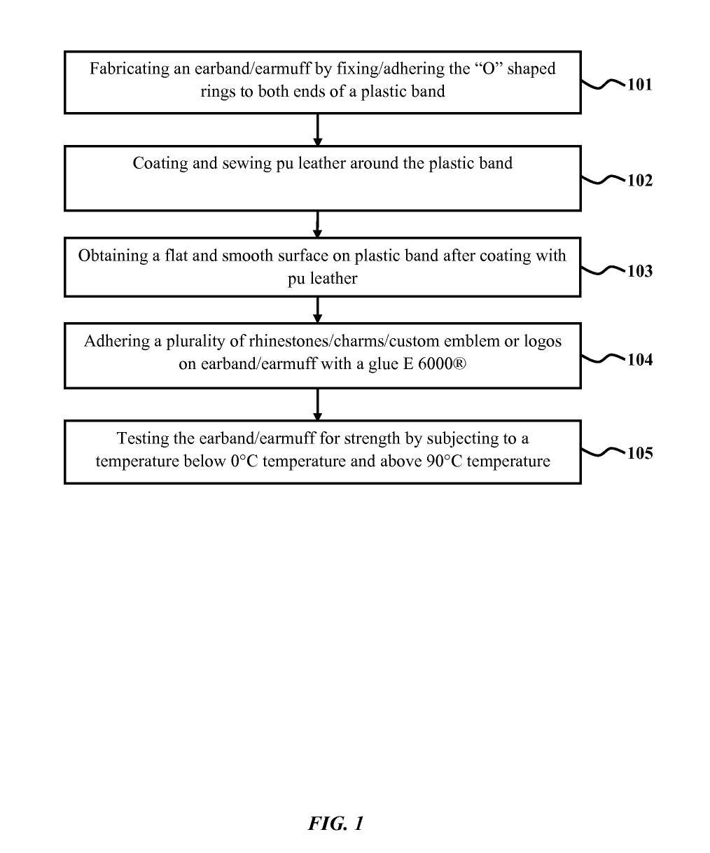

FIG. 1 illustrates a flowchart indicating a method for fabricating an earband/earmuff, according to an embodiment herein. With respect to FIG. 1, an earband/earmuff is fabricated by fixing or adhering the “O” shaped rings (101). Pu leather is coated and sewn around the plastic band (102). Further a flat and smooth surface is obtained on plastic band after coating with pu leather (103). a plurality of rhinestones/charms/custom emblem or logos is adhered on earband/earmuff with a glue (104). The earband/earmuff is tested for strength by subjecting to a temperature below 0° C. temperature and above 90° C. temperature (105).

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A method for fabricating an ear band/earmuff, the method comprises the steps of:
fixing or adhering the “O” shaped rings on two ends of a plastic band;
coating or sewing pu leather around the plastic band;
obtaining a flat and a smooth surface on the plastic band after coating with pu leather;
adhering a plurality of articles on the earband/earmuff with a glue;
curing the earband/earmuff after adhereing the plurality of articles with the glue for a preset curation time; and
subjecting the earband or earmuff to a plurality of temperature levels for testing a adhesive strength.

2. The method according to claim 1, wherein the glue is a craft adhesive or an article adhesive, and wherein the adhesive or glue is E6000®.

3. The method according to claim 1, wherein the plurality of articles are selected from a group comprising of a rhinestone, a charm, a customized emblem and a logo.

4. The method according to claim 1, wherein the ear band or earmuff is subjected to a predetermined temperature range, and wherein the temperature range is 0° C.-90° C.

5. The method according to claim 1, wherein the preset curation time after the glue or adhesive is applied to an article is 20 minutes.

6. The method according to claim 1, wherein the plurality of articles are adhered on the earband/earmuff with a thin adhesive film or a thick adhesive film.

7. The method according to claim 6, wherein a full curation time of a thin adhesive film is 24 hours, and wherein a full curation time of a thick adhesive film is 48-72 hours.

8. A glue or adhesive composition for adhering plurality of articles to an earband/earmuff comprises tetracholoroethylene, and wherein tetrachloroethylene is a cholorocarbon, and wherein the tetracholoethylene has a formula Cl₂C═CCl₂.

9. The composition according to claim 8, wherein the tetracholoroethylene is present in a predetermined quantity and wherein the predetermined quantity is 60-100 weight %.

10. The composition according to claim 8, wherein the glue composition has a sweet odour.

11. The composition according to claim 8, wherein the glue composition is non flammable.

12. The composition according to claim 8, wherein the glue composition has a density of 11.0±0.1 lbs/gal.

13. The composition according to claim 8, wherein the glue composition has a viscosity of 100,000 cps.

14. The composition according to claim 8, wherein a curation time after the glue or adhesive is applied to an article is 20 minutes,

15. The composition according to claim 8, wherein the glue is applied as a thin adhesive film or a thick adhesive film and wherein a full curation time of a thin adhesive film is 24 hours, and wherein a full curation time of a thick adhesive film is 48-72 hours.

16. The composition according to claim 6, wherein the glue composition is resistant to a plurality of dilute acids and bases.

17. The composition according to claim 16, wherein the dilute acids and bases are selected from a group comprising of an acetic acid (5% and 10% by volume), an ammonium hydroxide (3.4% by volume), an ammonium nitrate (50% by volume), a beer (3.2% by volume), a boric acid (3.1% by volume), a distilled water, a hydraulic oil, a lactic acid (3.8% by volume), a milk, a motor oil, a nitric acid (10% and 20% by volume), a oxalic acid (3.1% by volume), a phosphoric acid (30% and 60% by volume), a potassium hydroxide (3.4% by volume), a sodium carbonate (2.7% by volume), a sodium chloride (10%), a sulfuric acid (3% and 10% by volume) and a wine (20% alcohol by volume).
